(19) United States
(12) Patent Application Publication
(10) Pub. No.: US 2012/0041534 A1
(43) Pub. Date: Feb. 16, 2012

(54) STENT DELIVERY SYSTEM WITH INTEGRATED CAMERA

(75) Inventors: Claude Clerc, Marlborough, MA (US); Chris Benning, Lowell, MA (US); William C. Bertolino, Framingham, MA (US); John Lane, Manchester, NH (US); John Hutchins, North Attleboro, MA (US); Amie Fish, Northbridge, MA (US); Paul Aquilino, South Walpole, MA (US)

(73) Assignee: BOSTON SCIENTIFIC SCIMED, INC., Maple Grove, MN (US)

(21) Appl. No.: 13/206,850

(22) Filed: Aug. 10, 2011

Related U.S. Application Data
(60) Provisional application No. 61/372,277, filed on Aug. 10, 2010.

(51) Int. Cl. A61F 2/84; A61B 1/04

(52) U.S. Cl. 623/1.11; 600/109

(57) ABSTRACT

Various methods and devices are described for imaging a body lumen during delivery and deployment of a medical device. In one example, a delivery device includes at least one sheath, a prosthesis, an inner tubular member and at least two cameras to allow visualization of the prosthesis prior, during and after deployment without the use of an endoscope. The at least one sheath and/or the inner tubular member includes at least two cameras engaged to the delivery device.

This application claims the benefit of U.S. Provisional Application No. 61/372,277, entitled “STENT DELIVERY SYSTEM WITH INTEGRATED CAMERA,” by Claude Clerc, Chris Benning, Bill Bertilino, John Lane, John Hutchins, Amie Fish, and Paul Aquilino, and filed on Aug. 10, 2010, the entire contents of which being incorporated herein by reference.

### TECHNICAL FIELD

This disclosure relates to medical devices and, in particular, to delivery systems adapted for visualization of the deployment of a medical device.

### BACKGROUND

Stents and stent delivery assemblies are utilized in a number of medical procedures and situations, and as such their structure and function are well known. A stent is a generally cylindrical prosthesis that is introduced via a catheter into a lumen of a body cavity or vessel. The stent is introduced into the cavity or vessel with a generally reduced diameter and then is expanded to the diameter of the cavity or vessel. In its expanded configuration, the stent supports and reinforces the cavity/vessel walls while maintaining the cavity/vessel in an open, unobstructed condition.

A stent delivery catheter may be delivered over a guidewire. A guidewire is flexible and has a smaller diameter than a stent delivery catheter, and therefore can be inserted into the body cavity or vessel of interest first, over and along which a stent delivery catheter can follow. Typically, when deploying an endoscopically delivered stent in a body cavity of interest, a guidewire is introduced into the body cavity through a working lumen defined in an endoscope to ensure proper placement of the prosthesis. The guidewire is used to ensure that the device is properly positioned and the deployment device is maintained in the proper position during deployment of the prosthesis. A physician advances an endoscope and the guidewire removably received therethrough into the body cavity of interest while observing an image received from the distal end of the endoscope. Once the distal end of the guidewire reaches the position of interest, as observed by the endoscope, the endoscope can be withdrawn, leaving the guidewire in place. Thereafter, a stent delivery catheter is passed over the guidewire and the stent is deployed. To observe and ensure proper deployment of the stent, the endoscope can be passed along the side of the stent during deployment. In addition, for example, when applying a stent in a blood vessel, fluoroscopy (x-ray imaging of a moving object) is often used to ensure proper placement and deployment of the stent, as well known in the art.

### SUMMARY

In one example, the disclosure is directed to a prosthesis delivery device comprising at least one sheath, a prosthesis, an inner tubular member and at least two cameras. The at least one sheath removably covers the prosthesis therein. The at least one sheath comprises a distal end, a proximal end, an outer surface and a channel extending between the distal end and the proximal end. The channel defines an inner wall. The prosthesis extends in a compressed state within the channel. The inner tubular member slidably extends through the prosthesis, the inner tubular member comprising an elongated inner shaft with a distal tip at one end. The at least two cameras are engaged to the delivery device.

In another example, the disclosure is directed to a stent delivery device including at least one sheath, a stent and an inner tubular member. The at least one sheath removably covers the stent therein. The at least one sheath comprises a distal end, a proximal end, an outer surface and a channel extending between said distal end and said proximal end. The channel defines an inner wall. The stent extends in a compressed state within said channel. The inner tubular member slidably extends through the stent, and the inner tubular member comprises an elongated inner shaft with a distal tip at one end. The at least two cameras are engaged to the delivery device.

In another example, the disclosure is directed to a method for intraluminally positioning a prosthesis comprising providing a delivery device comprising at least one sheath removably covering a prosthesis therein, said at least one sheath includes a distal end, a proximal end, an outer surface and a longitudinal channel extending between said distal end and said proximal end, said channel defining an inner wall, said prosthesis extending in a compressed state within said longitudinal channel, an inner tubular member slidably extending through said prosthesis, said inner tubular member comprises an elongated inner shaft with a distal tip at one end, and at least two cameras engaged to said delivery device. The method further comprises activating said at least two cameras to provide images during positioning of said prosthesis, positioning said delivery device within a body lumen, and slidably retracting said at least one sheath relative to the inner tubular member to uncover said prosthesis and allow said prosthesis to radially expand against a wall of body lumen.

These and other features of the invention will be more fully understood from the following description of specific embodiments of the invention taken together with the accompanying drawings.

### BRIEF DESCRIPTION OF DRAWINGS

FIG. 1 is a schematic view of one example delivery system in accordance with various techniques of this disclosure.

FIG. 2 is a schematic view of another example delivery system in accordance with various techniques of this disclosure.

FIG. 3 is a schematic view of another example delivery system in accordance with various techniques of this disclosure.

FIG. 4 is a schematic view of another example delivery system in accordance with various techniques of this disclosure.

Corresponding reference characters indicate corresponding parts throughout the several views of the drawings.

### DETAILED DESCRIPTION

Endoscopes are commonly used to deliver stents into a body cavity. When delivering a stent in a body cavity of interest, a guidewire is introduced into the body cavity through a working lumen defined in an endoscope. An endoscope, however, has a diameter that is relatively large with respect to the body cavity or body lumen of interest. Thus, the use of an endoscope to deliver a guidewire (and hence a stent delivery catheter) becomes difficult in some applications. For example, esophageal stents, gastrointestinal (GI) stents, and pulmonary stents are fairly large thereby requiring a larger delivery system. Therefore, positioning an endoscope along the side of a stent to observe its proper deployment requires an even larger space, which is not always available. As mentioned above, a physician is generally required to use an endoscope to place a guidewire, remove the endoscope leaving the guidewire in place, reinsert the endoscope along the guide wire, and insert the stent over the guidewire.

Still further, use of fluoroscopy to confirm proper positioning of a guidewire and/or a stent is a relatively cumbersome procedure that requires additional safety mechanisms for the patients as well as the doctors and their assistants. As such, a need exists for a vision system that is integral with the stent delivery system to provide a device that deploys and provides imaging in a single device. Additionally, a need exists for a stent delivery system having imagining capabilities to allow visualization of stent prior, during and after deployment without the use of an endoscope. A need exists for a single device that provides visualization and deployment of a prosthesis without the required use of fluoroscopy and/or a separate endoscope. In general, this disclosure describes delivery devices and methods used to deploy various implants or prostheses, e.g., stents, where the delivery device includes a vision system that is integral to the delivery system, thereby reducing or eliminating the need for the physician to reintroduce an endoscope when delivering a stent.

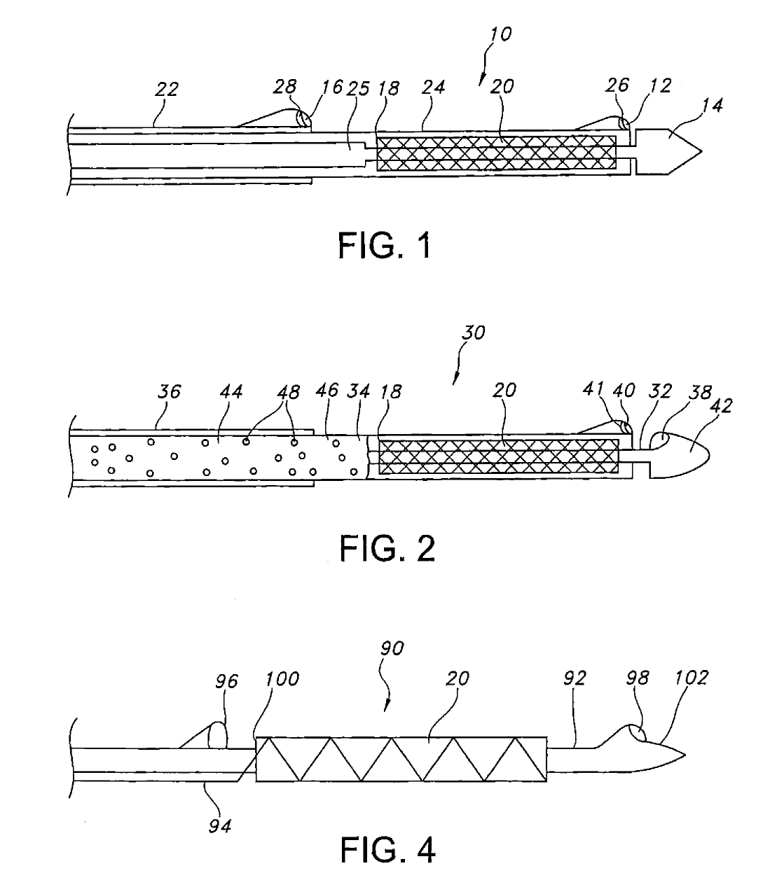

FIG. 1 is a schematic view of one example delivery system in accordance with various techniques of this disclosure. The delivery device 10 shown has two cameras, namely cameras 12, 16, that are engaged to the delivery device. The delivery device 10 may include a first or outer sheath 22, a second or middle sheath 24 and an inner member 25. In one example configuration, the cameras are engaged to the delivery device by being integrally formed and embedded into the delivery device, e.g., in one of the sheaths 22, 24 and/or inner member 25, by molding the cameras into the material of the delivery device during the manufacturing of the device. In another example configuration, the cameras are engaged to the delivery device by being fixedly attached, e.g., to one of the sheaths 22, 24 and/or inner member 25, by way of adhesives, screws, or other fasteners. The first camera 12 located close to the distal tip 14 of the delivery device 10 is integrally formed from and embedded into the middle sheath 24 of the delivery device 10. The first camera 12 allows for evaluation of the anatomy prior to stent release.

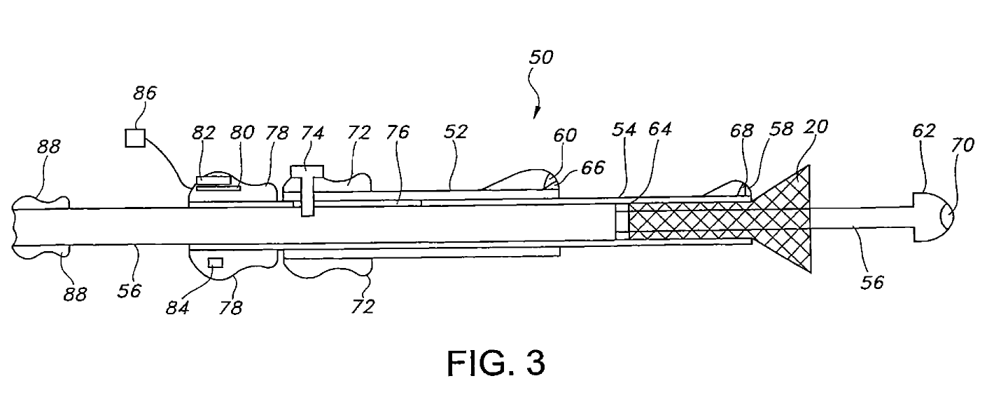

The second camera 16 located near the proximal end 18 of the stent 20 is integrally formed from and embedded into the outer sheath 22 of the delivery device 10. The second camera 16 allows for observation of the proximal end 18 of the stent 20 during stent deployment. After the stent 20 has been deployed, the first camera 12 can be used to confirm stent placement and re-inspect the anatomy, as shown in FIG. 3.

In the example configuration depicted in FIG. 1, inner member 25 does not include a camera. In addition, cameras 12, 16 include illumination devices 26, 28, respectively, to provide illumination within the lumen for enhanced visualization.

The cameras described in this disclosure may include an imaging chip, e.g., charge-coupled device (CCD) sensor or a complementary metal oxide semiconductor (CMOS) in nature, and a lens constructed with single or multiple optical elements. Additionally, the cameras may acquire an image through an imaging fiber bundles rather than directly. The images from the cameras are sent as imaging signals through hardwires or other signal transmitting members or wirelessly transmitted for reception and processing for display on an external device. The cameras of the present invention are of a size and shape driven by the mechanical attributes of the stent delivery system described. It is suggested that, in some examples, the cameras are miniature in nature (e.g., less than 4 millimeters in diameter or diagonal) with resolutions limited only by the state of the art of imaging arrays and lens construction, and lenses such as, but not limited to, micro-lenses and wafer-scale lenses are used. The cameras are positioned on the stent delivery device in such a way as to image specific areas of interest during navigation or stent deployment and therefore may have a primary direction of view at any angle. Camera lens parameters are likewise tuned at design to fulfill specific requirements of the application, e.g., field of view, depth of view, magnification, and the like.

The illumination device or system of the present invention provides light for the operation within a body lumen. The illumination device may include, but is not limited to, one or more light emitting diodes (LEDs), a fiber optic illumination guide for providing light from a light source, such as a laser or a white light source, and the like. Further, a lens may also be provided at the distal end of the illumination device to focus the illumination on the body lumen or tissue.

The light can be provided as a separate light source from the camera/camera processor or combined into a single piece of equipment. This equipment is located remotely from the stent delivery device and positioned as a matter of convenience to the practitioner. In the example configuration shown in FIG. 3, one or several additional connectors may be needed to provide the light from the separate light source. The light can also be produced by one or more LEDs located close to each camera. In this configuration, power is supplied to the LEDs via suitable electrical wires to provide simultaneous or independent control of LED light output.

One or more LEDs may also be located in the handle. In one example, the light may be transmitted to a location close to the camera via optical fibers. The optical fibers can form a single bundle, multiple bundles, or be incorporated evenly in the circumference of the middle sheath and/or outer sheath. The illumination device and/or camera may include, but is not limited to, an objective lens and fiber optic imaging light guide communicating with a practitioner, a camera, a video display, a sensor, such as a charge-coupled device (CCD) sensor or a complementary metal oxide semiconductor (CMOS) sensor, and the like. In any of the illumination configurations described, control of the light source or sources may be controlled manually or automatically through a camera processor driven by feedback control. Manual control of the illumination maybe coupled with automatic control of the camera pixel gains or automatic control of the illumination may be coupled with automatic camera pixel gain control.

FIGS. 2-4 are schematic views of three example delivery systems in accordance with various techniques of this disclosure. The devices of FIGS. 2-4 may include a camera or camera and an illumination system integrally formed and within the inner member and embedded therein. FIG. 2 shows delivery device 30 including an inner member 32, a middle sheath 34 and an outer sheath 36. The inner member 32 may include a first camera 38 located in the tip 42 which is directed towards the proximal end or a backwards viewing camera. It is contemplated that the camera can be located in a variety of positions such as shown in FIGS. 3-4. Additionally, this disclosure contemplates that the cameras described can be rotation cameras such that the cameras move/rotate to different positions/angles within its socket.

Referring now to FIG. 2, the first camera 38 allows for observation of the proximal end 18 of the stent 20 during stent deployment. After the stent 20 has been deployed, the first camera 38 can be used to confirm stent placement and re-inspect the anatomy. The inner member 32 may also include an illumination system (not shown).

In some example configurations, the middle sheath 34 may include an illumination system 44 and a second camera 40, as depicted in FIG. 2. The illumination system 44 is integrally formed and embedded into the outer surface of the middle sheath 34. The illumination system 44 may include a plurality of illumination devices 48 such as optical fibers that terminate at different locations on the external surface 46 of the middle sheath 34 to provide continuous illumination along the length of the delivery device. Illumination can be provided anywhere in the system, including inside the body of the catheter (catheter may be clear or opaque).

The second camera 40 allows for evaluation of the anatomy prior to stent release. As seen in FIG. 2, the second camera 40 may include an illumination system 41.

FIG. 3 is a schematic view of another example delivery system in accordance with various techniques of this disclosure. Generally speaking, FIG. 3 depicts a delivery device 50 that combines feature shown and described with respect to FIGS. 1 and 2, including cameras located in the middle sheath and outer sheath (FIG. 1) and a camera on the inner member (FIG. 2). More specifically, FIG. 3 depicts a delivery device having cameras 60, 58, and 70 integrally formed from and embedded into an outer sheath 52, middle sheath 54 and an inner member 56, respectively.

FIG. 3 depicts one example of the positioning and functioning of the cameras. In FIG. 3, the stent 20 is being deployed as the middle sheath 54 is retracted. The first camera 58 located close to the distal tip 62 of the delivery device 50 is integrally formed from and embedded into the middle sheath 54 of the delivery device 50. The first camera 58 allows for evaluation of the anatomy prior to stent release. The second camera 60 located near the proximal extremity 64 of the stent 20 is integrally formed from and embedded into the outer sheath 52 of the delivery device 50. The second camera 60 allows for observation of the proximal extremity 64 of the stent 20 during stent release. The inner member 56 may include third camera 70 on the distal tip 62. The third camera 70 is a forward-facing camera. The third camera 70 allows for evaluation of the anatomy prior to stent deployment and also upon removal of the delivery device from the body lumen. Each of the cameras 58, 60, 70 are integrally formed from and embedded into a respective sheath, e.g., sheaths 54, 52, 56, to provide a smooth exterior surface on the sheaths and minimize the overall diameter and size of each sheath and the delivery device as a whole. After the stent 20 has been deployed (not depicted), the first camera 58 and the second camera 60 can be used to confirm stent placement and re-inspect the anatomy.

Further, in some examples, cameras 58, 60 include illumination devices 66, 68, respectively, to provide illumination within the lumen. The distal tip 62 may also include an illumination device (not shown).

In the example configuration shown in FIG. 3, the distal handle 72 is connected to the outer sheath 52, the middle handle 78 is connected to the middle sheath 54 and allows for stent deployment when the middle sheath is retracted, and the proximal handle 88 is connected to the inner member 56. A pin 74, which can be removable, connects the outer sheath 52 to the inner member 56 to maintain the position of the camera 60 when the outer sheath 52 is pulled back. In the example shown in FIG. 3, a gap 76 in the middle sheath 54 allows motion of middle sheath 54 when the pin 74 is in place. In other examples, instead of the pin 74, the proximal handle 88 and the distal handle 72 may be linked by an external connector. The pin ensures that the relative position of the proximal handle and distal handle is fixed when the middle handle is moved. This can also be achieved by connecting the proximal handle 88 and the distal handle 72.

As seen in FIG. 3, the middle handle 78 may include a circuit board 80 to drive the cameras, a battery 82 to provide power to the cameras and illumination systems, an optional switch 84 to switch between cameras 60, 58 and 70, and a video connector 86. There is an electrical connector (not shown) between the middle handle 78 and distal handle 72 for the camera and illumination system. If the delivery device is disposable, the battery 82 can be removed from the handle to dispose of the device. In some examples, the battery 82, circuit board 80, switch 84, and video connector 86 may be located in several different handles. Power is supplied from a power source to each of the cameras and illumination systems by various means, including wires or conductive material embedded into the particular sheath into which a respective camera and illumination system is embedded.

FIG. 4 is a schematic view of another example delivery system in accordance with various techniques of this disclosure. In particular, FIG. 4 shows a two camera system including first camera 96 located at the proximal end 100 of the stent and second camera 98 located closer to the distal tip 102, and a stent 20 is held in place on the delivery device 90 with a crochet suture 94. Although two cameras are depicted, in some example configurations, a single camera may be installed.

The cameras 96, 98 in FIG. 4 are mounted on a delivery device 90 that may include a single inner member 92, such as Boston Scientific Corp's Ultraflex™ Stent Delivery System. In the example shown in FIG. 4, the cameras 96, 98 are integrally formed from the inner member 92 and embedded therein. The inner member 92 may be a solid rod or a hollow tube that allows for the passage of a guidewire to maintain a position of delivery device during deployment of a prosthesis, e.g., stent 20, and/or to facilitate the accurate placement of the prosthesis, the passage of other material such as injecting contrast medium, or the passage of wires to supply power and video signals to/from the cameras. Further, the inner member 92 may include various markings along the length to provide a ruler or means of measuring the distance the device has travelled within the lumen. The delivery device may also include a means to steer the distal tip 102 to allow several degrees of liberty, e.g., two degrees, similar to a SpyScope® Access and Delivery Catheter, available from Boston Scientific, to facilitate device insertion. Further, the distal tip 102 may include a camera in various positions and integrated at various positions along the inner member 92, such as a forward-facing camera as shown in FIG. 3.

Additionally, it is contemplated that the distal tip of the present invention may be transparent and may include multiple cameras therein. Further, the camera and illumination devices may be located side-by-side, or at different locations along the circumference of the inner member, middle sheath and/or outer sheath. It is further contemplated that the inner member, middle sheath and/or outer sheath can rotate independently of each other to allow for better visualization.

In another aspect, this disclosure is directed to a method for delivering a stent 20 into a body lumen or a method of use is provided. The device 10, 30, 50, 90 may be used for various applications such as esophageal stenting, colonic stenting, pulmonary stenting, urinary stenting, for various applications for orifice transluminal endoscopic surgery (NOTES), biopsy procedures and the like. The method of use includes providing a delivery device 10, 30, 50, 90, the device 10, 30, 50, 90 includes at least one sheath or stent retaining member to retain the prosthesis, such as a stent, in a compressed state until delivery, and an inner member 25 and at least one camera and/or illumination system located on at least one sheath, or located on the sheath and inner member; and a prosthesis or stent 20. The at least one sheath has a proximal end, a distal end, an outer wall and a longitudinal channel through the sheath defining an inner wall of the sheath and the stent 20 is juxtaposingly disposed to a distal portion of the inner wall and an inner member slidably disposed within the channel. The camera is activated to provide imaging during the delivery of the stent and the illumination system is activated to provide illumination within the lumen during the deployment process. The sheath is advanced through the lumen until properly positioned. Once the delivery device 10, 30, 50, 90 is positioned for deployment, the stent 20 may be released from the endoscopic stent delivery device 10, 30, 50, 90 by retracting the elongate sheath to release the stent 20 from the delivery device 10, 30, 50, 90 and/or by advancing the inner member to push the stent 20 out of the delivery device 10, 30, 50, 90. The cameras provide imaging throughout the deployment of the stent 20 to verify accuracy and placement of the stent. The step of providing the endoscopic stent delivery device 10, 30, 50, 90 may further include a step of loading the stent 20 within the distal portion of the inner wall of the endoscope 10, 30, 50, 90. The method may further include radially compressing the stent 20 prior to loading the stent 20 within the distal portion of the inner wall of the endoscope 10, 30, 50, 90.

Additionally, the method of use may include selecting the proper prosthesis, e.g., esophageal stents, gastrointestinal (GI) stents, and pulmonary stents, according to the patient anatomy and disease progression; loading the desired prosthesis into the delivery device 10, 30, 50, 90 or selecting a pre-loaded delivery device 10, 30, 50, 90 including the proper prosthesis; connecting the delivery device to external capital equipment to supply power and necessary external elements to the device; introducing the device through the desired orifice and extending the device through a lumen to the location for deployment; confirming proper positioning by direct visual confirmation and exploring the lumen and/or stricture to ensure proper placement of prosthesis, e.g., the esophago-gastroenoscopy (EGO) is performed by the device; measuring the stricture and recording the measurements; advancing a guidewire into the invention through the stricture; deploying the prosthesis by pulling back on the sheath while the physician watched the deployment under direct visualization by the cameras; ensuring proper placement of the prosthesis by direct visualization once the prosthesis has been deployed; removing the device from the lumen. Additionally, the camera and/or illumination system may be attached to the device prior to introducing the device with the lumen.

Furthermore, any of the above-described viewing devices and/or illuminating devices may be disposed on or within or in conjunction with any of the above-described any of the above-described components. Further, the viewing device and the illuminating device may be disposed on different components of the present invention.

While the invention has been described by reference to certain preferred embodiments, it should be understood that numerous changes could be made within the spirit and scope of the inventive concept described. Accordingly, it is intended that the invention not be limited to the disclosed embodiments, but that it have the full scope permitted by the language of the following claims.

Various aspects of the disclosure have been described. These and other aspects are within the scope of the following claims.

1. A prosthesis delivery device comprising:
at least one sheath removably covering a prosthesis therein, said at least one sheath comprising a distal end, a proximal end, an outer surface and a channel extending between said distal end and said proximal end, said channel defining an inner wall;
said prosthesis extending in a compressed state within said channel;
an inner tubular member slidably extending through said prosthesis, said inner tubular member comprising an elongated inner shaft with a distal tip at one end; and
at least two cameras engaged to said delivery device.

2. The delivery device of claim 1, wherein said at least one sheath includes one of said two cameras integrated into said at least one sheath and formed from said outer surface.

3. The delivery device of claim 1, wherein said inner tubular member comprises at least one of said at least two cameras attached to said elongated inner shaft.

4. The delivery device of claim 3, wherein one of said at least two cameras is attached to said distal tip.

5. The delivery device of claim 3, wherein one of said at least two cameras is attached to said distal tip and one of said at least two cameras is attached to said outer surface.

6. The delivery device of claim 5, wherein said at least one sheath is a wrap.

7. The delivery device of claim 1, wherein said at least one sheath comprises a first sheath and a second sheath, said first sheath comprises a first distal end, a first proximal end, a first outer surface and a first channel extending between said first distal end and said first proximal end, said first channel defining a first inner wall, said second sheath comprises an elongated shaft, a second proximal end, a second distal end, a second outer surface and a second longitudinal channel through the elongate second shaft defining a second inner wall of the second sheath, said second sheath extends within said channel, said prosthesis extending within said second channel.

8. The delivery device of claim 7, wherein said stent is juxtaposingly disposed to a distal portion of the second inner wall.

9. The delivery device of claim 8, wherein one of said at least two camera is attached to said second sheath and formed from said second outer surface.

10. The delivery device of claim 9, wherein one of said at least two camera is attached to said first sheath and incorporated into said first outer surface, and said at least two cameras are located in proximity to said first distal end and said second distal end.

11. The delivery device of claim 10, wherein one of said at least two cameras is attached to said distal tip of said inner tubular member.

12. The delivery device of claim 1, further comprising an illumination system integrated into said delivery system adjacent each of said at least two cameras.

13. The delivery device of claim 1, further comprising an illumination system integrated into said second outer surface of said second sheath.

14. The delivery device of claim 13, wherein said illumination system comprises an optical fiber along the length of said second sheath and said optical fiber terminating at said second outer surface.

15. A stent delivery device comprising:
at least one sheath removably covering a stent therein, said at least one sheath includes a distal end, a proximal end, an outer surface and a channel extending between said distal end and said proximal end, said channel defining an inner wall;
said stent extending in a compressed state within said channel;
an inner tubular member slidably extending through said stent, said inner tubular member comprising an elongated inner shaft with a distal tip at one end; and
at least two cameras engaged to said delivery device.

16. A method for intraluminally positioning a prosthesis comprising:
providing a delivery device comprising at least one sheath removably covering a prosthesis therein, said at least one sheath includes a distal end, a proximal end, an outer surface and a longitudinal channel extending between said distal end and said proximal end, said channel defining an inner wall, said prosthesis extending in a compressed state within said longitudinal channel, an inner tubular member slidably extending through said prosthesis, said inner tubular member comprises an elongated inner shaft with a distal tip at one end, and at least two cameras engaged to said delivery device;
activating said at least two cameras to provide images during positioning of said prosthesis;
positioning said delivery device within a body lumen; and
slidably retracting said at least one sheath relative to the inner tubular member to uncover said prosthesis and allow said prosthesis to radially expand against a wall of body lumen.

17. A method according to claim 16, wherein said step of providing comprises providing an illumination system attachable to said delivery device, the method further comprising activating said illumination system to provide illumination within said body lumen during positioning of said prosthesis.

18. The method according to claim 16, wherein said step of activating comprises supplying power to said at least two cameras.

19. The method according to claim 16, further comprising advancing the inner member in an opposite direction of said at least one sheath to push said prosthesis out from said delivery device.

20. The method according to claim 16, wherein one of said at least two cameras is integrally formed from said at least one sheath and another of said at least two cameras is integrally formed from said inner member.
